(19) United States
(12) Patent Application Publication
(10) Pub. No.: US 2019/0345530 A1
(43) Pub. Date: Nov. 14, 2019

(54) SPECTROMETER COMPATIBLE VACUUM AMPOULE DETECTION SYSTEM FOR RAPIDLY DIAGNOSING AND QUANTIFYING VIABLE BACTERIA IN LIQUID SAMPLES

(71) Applicant: AMT Scientific LLC, Halethorpe, MD (US)

(72) Inventors: He Zheng, Halethorpe, MD (US); Shengjun Zhang, Halethorpe, MD (US)

(21) Appl. No.: 16/136,414

(22) Filed: Sep. 20, 2018

Related U.S. Application Data
(63) Continuation-in-part of application No. 15/973,619, filed on May 8, 2018.

(51) Int. Cl. C12Q 1/04; G01N 21/33; C12Q 1/06; G01N 21/78; B01L 3/00

(52) U.S. Cl. CPC C12Q 1/045; G01N 21/33; C12Q 1/06; G01N 2021/335; B01L 3/50; B01L 3/527; G01N 21/78

(57) ABSTRACT

A vacuum ampoule detection system and a method detect and quantify viable bacteria in liquid samples. The vacuum ampoule that includes a supporting medium, a selective reagent, and a detecting reagent are useful in the rapid detection and quantification of viable heterotrophic bacteria in liquid samples. The vacuum ampoule detection system is suitable for the detection of total bacteria, E. coli, or total coliform, etc. The vacuum ampoule detection system is also compatible with spectrometer for visible light, UV light and fluorescence which can give more accurate analysis of the concentration of bacteria in the liquid samples.

### CROSS-REFERENCE TO RELATED APPLICATION

This application is a continuation-in-part of U.S. Ser. No. 15/973,619, filed on May 8, 2018, the entire disclosures of which are incorporated herein by reference.

### TECHNICAL FIELD

The disclosed subject matter relates generally to the field of rapid diagnosis of bacteria. More specifically, the present disclosure provides a self-filling vacuum ampoule system and a method to detect and quantify the bacteria.

### BACKGROUND

Bacterial contamination of drinking water supplies can cause gastrointestinal disease, impairments of cells of the digestive tract and organs, and life-threatening infections in people with depressed immune systems (EPA, 2011). In the case of human health, infection by gram-negative bacteria, such as Escherichia coli (E. coli) can cause urinary tract infection (UTI). Alternative approaches for detecting total bacteria in liquid samples have been developed in the past few years. The dip-slide method has been approved by Environmental Protection Agency (EPA) (Federal Register 40 CFR Parts 141 and 143) that can give a semi-quantitative estimation of total bacteria in sample in 24-48 hours. However, because the volume of liquid analyzed is unrepresentative and not repeatable (˜1 mL), the accuracy and consistency of the dip-slide method are fairly low.

Adenosinetriphosphate Bioluminescence Assay (ATPmetry) is an easy-to-operate method and can give results very quickly. However, ATPmetry has the same issue as the dip-slide method that the sample volume is very small (˜100 μL) that is not representative. In addition, the reagents used in ATPmetry method require low temperature conservation that is not convenient for the field test. EPA has covered several PCA-based techniques to detect total bacteria in drinking water (EPA, 2011). Although the PCA-based techniques methods described above are highly sensitive and informative, they require specialized laboratory equipment, qualified personnel and have a high cost. Plate counting is a traditional yet very accurate method to detect total bacteria in liquid samples. The disadvantage of plate counting is that it requires specialized laboratory equipment and qualified personnel to perform the test. In addition, plate counting often requires a relatively long time to get results.

None of these existing methods is at one and the same time accurate, rapid, usable in the field and cost effective. Therefore, there still exists a strong demand for a novel method for the detecting total bacteria in liquid samples having all the qualities defined previously.

### SUMMARY

In view of the foregoing, the present disclosure pertains to providing a diagnostic device, system, and a method that enable detection or quantification of a viable bacteria in a liquid sample in a quick and specific manner, without requiring complicated processes or equipment. Also, disclosed and recited herein is a vacuum ampoule viable bacteria detection system providing an all-in-one rapid detection test without any sophisticated laboratory equipment and further laboratory test. Further, the present disclosure provides a method and a composition using visible color change of the liquid sample that is capable of indicating a viable cell density of the bacteria in CFU/mL (colony formation unit per milliliter) ranging from <10 to 10⁸ CFU/mL. However, problems to be solved by the present disclosure are not limited to the above-described problems.

According to an exemplary embodiment of the present disclosure, a self-filling vacuum ampoule detection system to quantify viable bacteria in a liquid sample may include a vacuum ampoule, which includes a supporting medium, wherein the supporting medium comprises nutrients for culture bacterial species in the liquid sample; at least one selective reagent to inhibit a growth of interference microbial species in the liquid sample; and a detection reagent to quantify an amount of bacterial species in the liquid sample. When the vacuum ampoule is self-filled with the liquid sample and the viable bacteria is present in the liquid sample and react with the detection reagent, the self-filled vacuum ampoule may be configured to change color.

According to another exemplary embodiment of the present disclosure, a method to quantify viable bacteria in a liquid sample may include self-filling the liquid sample into a vacuum ampoule containing a supporting medium, at least one selective reagent, and a detection reagent; mixing the self-filled vacuum ampoule with the supporting medium, the at least one selective reagent, and the detection reagent in the self-filled vacuum ampoule; incubating the self-filled vacuum ampoule at about 37° C.; during the incubating, observing a change of color of the self-filled vacuum ampoule, wherein the self-filled vacuum ampoule is configured to change color when the viable bacteria is present and react with the detection reagent; measuring an elapsed time started from beginning of incubation to time at the change of color of the self-filled vacuum ampoule; and determining a viable cell density of the bacteria in the liquid sample based on the elapsed time.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an example of a self-filling vacuum ampoule.

FIGS. 2A and 2B illustrate the results of the total viable bacteria ampoule test for the negative control and positive control.

FIG. 3 is a graph showing the relationship of the reaction time and bacterial density for the total viable bacteria ampoule test.

FIG. 4 shows the visual results of the total bacteria ampoule test from low to high bacterial densities.

FIG. 5 shows the absorbance spectrum of the total viable bacteria ampoule test with and without TTC.

FIG. 6 shows the absorbance spectrum of the total viable bacteria ampoule test over time.

FIG. 7 illustrates the results of the E. coli ampoule test for the negative control and positive control.

FIG. 8 is a graph showing the relationship of the reaction time and bacterial density for the E. coli ampoule test.

FIG. 9 shows the fluorescent spectrum of the E. coli ampoule test with and without 4MUG.

FIG. 10 shows the fluorescent spectrum of the E. coli ampoule test over time.

FIG. 11 is illustrations of processes to conduct a bacteria test using the self-filling vacuum ampoule for detection and estimation of cell density of viable bacteria in liquid sample.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

Unless otherwise defined, all terms (including technical and scientific terms) used herein have the same meaning as commonly understood by one having ordinary skill in the art to which this invention belongs. It will be further understood that terms, such as those defined commonly used in dictionaries, should be interpreted as having a meaning that is consistent with their meaning in the context of the relevant art and the present disclosure and will not be interpreted in an idealized or overly formal sense unless expressly so defined.

An in-vitro diagnostic device, system, and a method for the detection of viable bacteria (e.g. viable heterotrophic bacteria) in liquid sample rapidly (e.g. within 24 hours) are provided. In one embodiment, a vacuum ampoule detection system provides an all-in-one rapid detection test without any sophisticated laboratory equipment and further laboratory test. The vacuum ampoule detection system is suitable for the detection of total bacteria, E. coli, or total coliform, etc. A method described herein can be performed in the field completely by personnel without specific microbiology training. The method and compositions described herein is based on visible color change of liquid sample and capable of indicating a cell density of viable bacteria in the liquid sample in CFU/mL (colony formation unit per milliliter) ranging from about <10 to about 10⁸ CFU/mL. The positivity of the viable bacteria in liquid sample can also be measured with a visible light, UV light and fluorescence spectroscopy for more accurate analysis of bacteria concentration in the liquid sample. As a result, the method according to the disclosed embodiments enables detection or identification and quantification of a bacterium in a quick, accurate and specific manner, without requiring complicated processes or equipment.

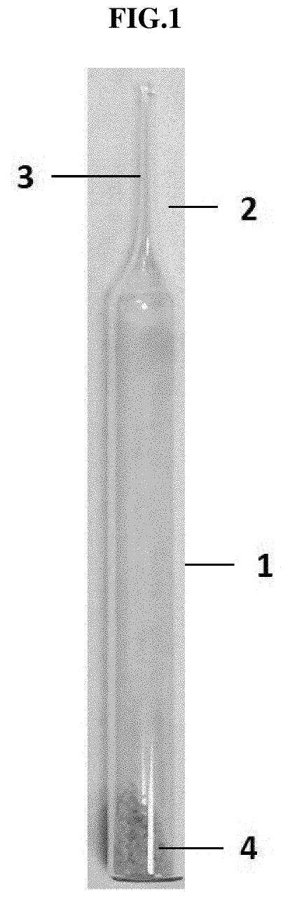

Example structures applicable in any system herein are described. FIG. 1 is an example of a vacuum ampoule bacteria detection system including a self-filling vacuum ampoule. As shown in FIG. 1, the vacuum ampoule total bacteria detection system includes a glass self-filling ampoule and an enclosed supporting medium and chemical indicator. According to one example of the glass self-filling ampoule, a cylindrical glass ampoule body 1 has a length of 3.5 inches and a diameter of 0.45 inches. The ampoule neck 2 has a length of 1.5 inches. The etching point 3 of the cylindrical glass ampoule is the middle of the ampoule neck (e.g. 0.75 inches from the tip of the ampoule neck). Supporting medium, selective reagent(s) and detection reagent(s) 4 are enclosed in the glass vacuum ampoule 1.

In some embodiments, a vacuum ampoule can take in about 7 mL liquid sample upon breaking a glass tip of the vacuum ampoule. A supporting medium that provides nutrients for bacterial culturing, selective reagent(s) that inhibit the growth of non-interested microbial species, and detection reagent(s) that indicates the presence of bacteria and provides indication of the cell density of the bacteria are included in the vacuum ampoule. The vacuum ampoule detection system is suitable for detection and quantification of total bacteria, E. coli, or total coliform, etc. in a liquid sample.

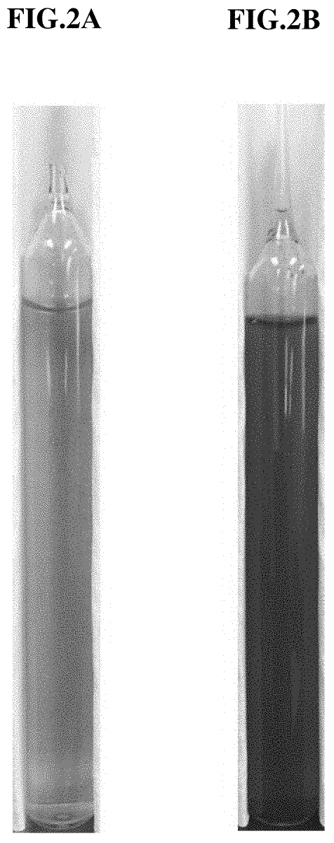

As an example, the total viable bacteria ampoule contains nutrients for bacterial culturing including about 1-20% of yeast extract, about 10-40% of peptone, about 10-40% of sodium chloride, about 1-10% of lab-lemco powder, and a detection reagent including about 0.1-1% of 2,3,5-triphenyltetrazolium chloride (TTC). FIGS. 2A and 2B show results of the total viable bacteria ampoule test by a positive control and a negative control. The terms “control” or “control sample” refers to any sample appropriate to the detection technique employed. For the negative control, the total viable bacteria ampoule contains about 1-20% of yeast extract, about 10-40% of peptone, about 10-40% of sodium chloride, about 1-10% of lab-lemco powder, and about 0.1-1% of 2,3,5-triphenyltetrazolium chloride (TTC), and the total viable bacteria ampoule is self-filled with about 7 mL of sterile milli-Q water. For the positive control, the total viable bacteria ampoule contains 1-20% yeast extract, 10-40% peptone, 10-40% sodium chloride, 1-10% lab-lemco powder, and 0.1-1% 2,3,5-triphenyltetrazolium chloride (TTC), and is self-filled with about 7 mL 10⁸ CFU/mL mixed viable cell suspension of E. coli ATCC 25922 and Staphylococcus aureus (S. aureus) ATCC 25923. The ampoules for the negative control and positive control are incubated at 37° C. for 1.5 hours.

FIGS. 2A and 2B show digital photographs representing the real testing results for the negative control and positive control of the total viable bacteria ampoules without modification. After around 24 hours of incubation, a photo image of the negative control is captured and indicates yellow color as shown in FIG. 2A. On the other hand, a photo image of the positive control captures a color change into pink-red as shown in FIG. 2B. The color change is recorded as bacterial growth and presence of the bacteria. In the negative control, since there are no bacteria inoculated, the ampoule remains clear and yellow color. In the positive control, the inoculated bacteria may amplify within certain time (depending on the inoculation concentration of the bacteria). The ampoule for the positive control looks cloudy and become pink-red color due to the enzymatic reaction between TTC (reagent) and bacteria.

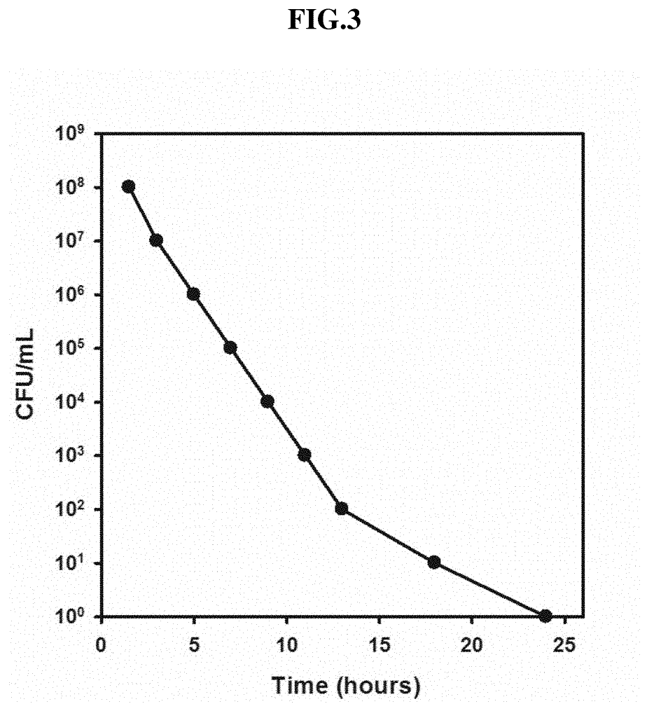

FIG. 3 is a graph illustrating the semi-quantitative results of the total viable bacteria vacuum ampoule. As can be seen in the graph, a negatively proportional correlation between the elapsed time (x-axis) of the appearance of the pink-red color and a viable cell density (CFU/mL) of E. coli ATCC 25922 and S. aureus ATCC 25923 in the liquid samples (y-axis) is presented. The nine ampoules used in the semi-quantitative tests are prepared by filling mixed viable cell suspension of E. coli ATCC 25922 and S. aureus ATCC 25923 in the total viable bacteria ampoule containing 1-20% of yeast extract, 10-40% of peptone, 10-40% of sodium chloride, 1-10% of lab-lemco powder, and 0.1-1% of 2,3,5-triphenyltetrazolium chloride (TTC). The above nine ampoules are filled with different cell densities of mixed viable cell suspension of E. coli ATCC 25922 and S. aureus ATCC 25923 and are incubated 37° C. until the nine ampoules show positivity of the inoculated bacteria (i.e. until pink-red color is shown). Each point on the graph represents test results of each of the nine ampoules. A viable cell density (CFU/mL) of E. coli ATCC 25922 and S. aureus ATCC 25923 in the liquid sample (y-axis) is measured using plate count taking the liquid sample from the original bacterial culture. Several dilutions are performed to prepare different cell densities for respective nine ampoules. Then, the elapsed time for respective nine ampoules are recorded which starts from the time of inoculating bacteria into respective ampoules to the time of the first appearance of pink-red color. The elapsed time for respective nine ampoules is plotted in x-axis of the graph in FIG. 3. The elapsed time of the tests ranges from 1.5 hours to 24 hours. Biological triplicates are prepared for each condition. Data variance is +/−1 hour.

The semi-quantitative results of the total viable bacteria vacuum ampoule in FIG. 3 are used to estimate cell density in the test samples by measuring the elapsed time of the samples and using the graph in FIG. 3. For example, a method for the detection of total viable bacteria in liquid sample begins by preparing the liquid test sample in the total viable bacteria vacuum ampoule, incubating the total viable bacteria vacuum ampoule, and checking the color change periodically (e.g. every hour) until pink-red color for respective samples is shown. Then, the time of the color change appearance for the samples are recorded and the bacteria density (i.e. viable cell density (CFU/mL)) in the tested liquid sample are estimated using the negatively proportional correlation between the elapsed time (x-axis) of the appearance of the pink-red color and a viable cell density (CFU/mL) of E. coli ATCC 25922 and S. aureus ATCC 25923 in the liquid samples (y-axis) in FIG. 3. By finding a particular Y value from the graph in FIG. 3 corresponding to the recorded elapsed time for the self-filled vacuum ampoule total bacteria detection system, the viable cell density (CFU/mL) of the bacteria in the liquid sample is estimated. This estimation method is capable of indicating a viable cell density of the bacteria in CFU/mL (colony formation unit per milliliter) ranging from <10 to 10⁸ CFU/mL.

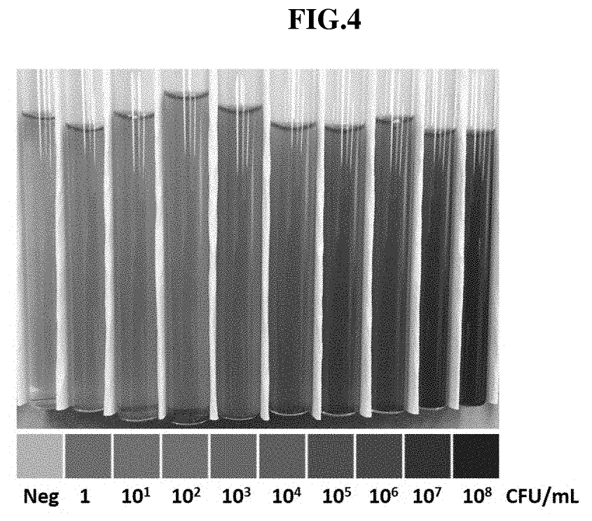

FIG. 4 shows the color of samples based on a visible color change detection after the total viable bacteria ampoule tests. The negative control as prepared in a same way with the negative control used in FIG. 2A is used for the visible color change detection. Further, the positive controls are prepared with different nine cell densities for respective nine ampoules for the visible color change detection as same to the nine ampoules used in the semi-quantitative tests shown in FIG. 3. As can be seen in visible color change detection of the samples, a positive proportional correlation between the intensity of the pink-red color and viable cell density (CFU/mL) of E. coli ATCC 25922 and S. aureus ATCC 25923 in the liquid samples is presented. The viable cell density is the initial inoculated cell density from original bacteria culture and calculated based on plate counting. The intensity of the pink-red color can be measured using visible color change detection or a UV spectrometer. Ampoules are incubated at 37° C. for 24 hours. Biological triplicates are prepared for each condition.

FIG. 4 shows digital photographs representing the real test results for the negative control and nine positive controls without modification. Then, color gradient references in FIG. 4 are generated for respective negative control and nine positive controls based on the digital photographs of the negative control and nine positive controls using image processes. The method of image processing for the digital photographs as known by one of ordinary skill in the art may be employed. The color gradient references are used as standards for visible color change detection to estimate cell density in the samples by comparing the color change of the samples to the color gradient references after incubating the samples. For example, a method for the detection of viable bacteria in liquid sample begins by preparing the liquid test sample the in the total viable bacteria vacuum ampoule and incubating the total viable bacteria vacuum ampoule for 24 hours. Then, a color change to the pink-red color of the liquid test sample is observed and the observed pink-red color (i.e. color intensity) is compared to the color gradient references to estimate what is the bacteria density (i.e. viable cell density (CFU/mL)) in the tested liquid sample. This estimation method is capable of indicating a viable cell density of the bacteria in CFU/mL (colony formation unit per milliliter) ranging from <10 to 10⁸ CFU/mL.

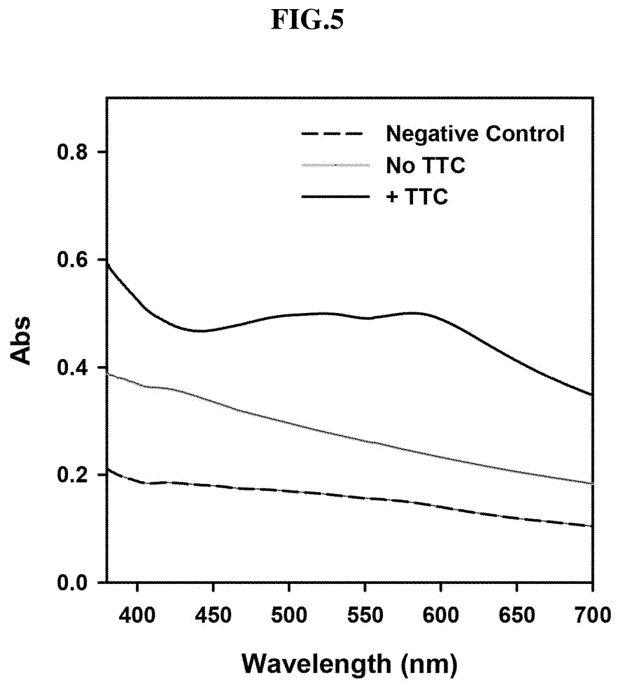

In another embodiment, the total viable bacteria test result can be quantified using a UV spectrometer. FIG. 5 shows the UV absorbance spectrums of the total viable bacteria ampoule tests with and without TTC and negative control. Three samples are prepared for the UV spectrometer. For the first sample, the total viable bacteria test is performed using 10⁶ CFU/mL as the initial inoculation concentration of E. coli ATCC 25922 and S. aureus ATCC 25923 with the presence of TTC. The ampoule for the first sample includes same nutrients for bacterial culturing used in the semi-quantitative tests in FIG. 3. For the second sample, the negative control, in which no bacteria is inoculated, is prepared. Other than the existence bacteria inoculation, the nutrients for bacterial culturing and the reagents (TTC) in the ampoule for the negative control are same with the fist sample. Instead of the bacteria inoculation, sterile milli-Q water with a neutral pH (7.0) is added to the negative control. For the third sample, the “No TTC” group” is prepared by applying same bacterial concentration (i.e. 10⁶ CFU/mL as the initial inoculation concentration) to the first sample but without adding the detection reagent, TTC. Then, the UV spectrometer measurement is performed for three samples at room temperature. The solution with only same nutrient components is used as the negative control with a neutral pH (7.0). Biological triplicates are prepared for each condition. The absorbance spectrums of the three samples are recorded between 300 nm and 800 nm by, for example, a DU 800 spectrophotometer (Beckman Coulter DU800). Other means of UV spectrometer may be employed by those having ordinary skill in the art.

In FIG. 5, an absorbance peak is observed at about 581 nm in the “+ TTC” group, but is not observed in “No TTC” and not in “Negative Control” groups. These results indicate that the UV spectrometer can be used to quantitatively measure the positivity (i.e. pink-red color) of total viable bacteria test result. The UV spectrometer can detect the appearance of pink-red color of total viable bacteria test result quicker than the visual color detection. For example, for 10⁶ CFU/ml initial bacteria cell density in the tested liquid sample, it normally takes 5 hours to observe visually the appearance of the pink-red color. On the other hand, if an absorbance at 580 nm is continuously measured via the UV spectrometer after initial bacterial inoculation (10⁶ CFU/ml), a notable peak at 580 nm absorbance can be observed about an hour before observing the visual color change.

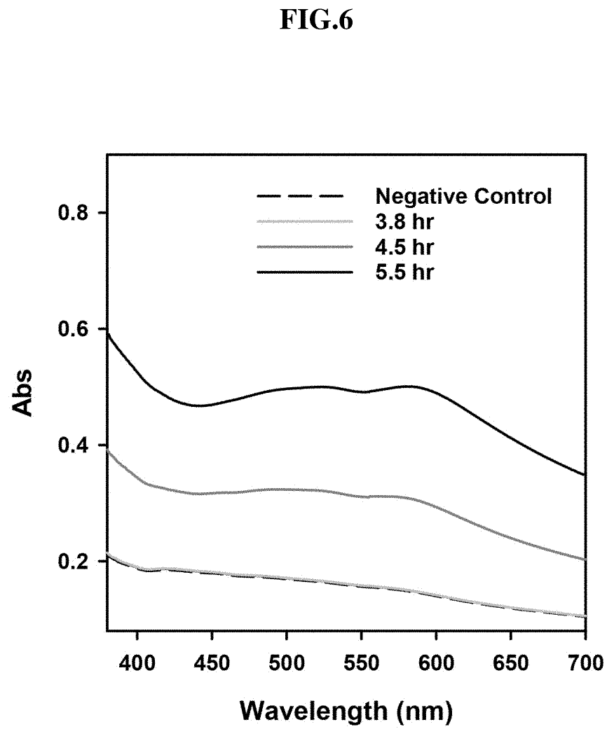

The appearance of the pink-red color is a result of an accumulative effect, that is, as a reaction between bacteria and TTC (detection reagent) continues, the color of the total viable bacteria test ampoule will become darker. This is confirmed by measurement of the UV absorbance spectrum over time as shown in FIG. 6. Two samples (i.e. “+ TTC” group, and “Negative Control” group) used in the above UV absorbance spectrums in FIG. 5 are prepared. Then, the UV spectrometer measurement for the “+ TTC” group is performed with different incubation time (e.g. 3.8 hours, 4.5 hours, and 5.5 hours). The condition for the UV spectrometer measurement is same with the UV spectrometer measurement in FIG. 5. With same initial bacterial inoculation (i.e., 10⁶ CFU/mL as the initial inoculation concentration of E. coli ATCC 25922 and S. aureus ATCC 25923 with the presence of TTC), the absorbance peak at 581 nm grows as the incubation time increases. On the other hand, the UV spectrometer measurement is performed for the “Negative Control” group after 5.5 hours of the incubation time but an absorbance peak at about 581 nm is not observed. These results indicate that the absorbance of 580 nm is only correlated to the reaction between bacteria and TTC, and as the incubation time increases, the intensity of the pink-red color increases.

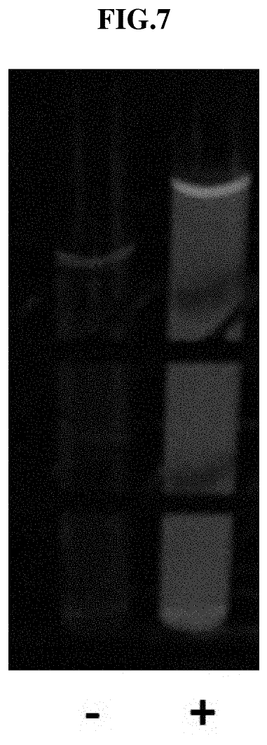

As another exemplary embodiment, the detection of E. coli is determined by examining fluorescence in the long-wave UV range, which fluorescence may indicate the presence of the inoculation of E. coli culture, and the absence of fluorescence may indicate the absence of the inoculation of E. coli culture in the test sample. The E. coli ampoule is prepared and contains nutrients for bacterial culturing including about 1-20% of yeast extract, about 10-40% of peptone, about 10-40% of sodium chloride, about 1-10% of lab-lemco powder, and a detection reagent including about 0.1-1% of 4-Methylumbelliferyl-β-D-glucuronide hydrate (4-MUG). Then, a positive control and a negative control using the E. coli ampoule are prepared. For the negative control (“−”), the E. coli ampoule of the above is filled with about 7 mL of sterile milli-Q water instead of mixed viable cell suspension of E. coli ATCC 25922. For the positive control (“+”), the E. coli ampoule of the above is self-filled with about 7 mL 10⁸ CFU/mL mixed viable cell suspension of E. coli ATCC 25922. The self-filled E. coli ampoule is incubated at 37° C. for 24 hours. Next, the negative control and the positive control are examined under the long-wave UV light. The fluorescence examination results for the negative vs positive of the E. coli ampoule tests are shown in FIG. 7. In FIG. 7, strong blue fluorescence under the long-wave UV light is observed in the positive control (“+”) but not in the negative control (“−”). 4-MUG, which is a detection reagent for the E. coli bacteria, is a non-fluorescent substance, and E. coli bacteria can produce beta-D-glucuronidase which is able to hydrolyze 4-MUG into 4-methylumbelliferyl moiety with blue fluorescence under long-wave UV light. Digital photographs in FIG. 7 represent the real testing results without modification.

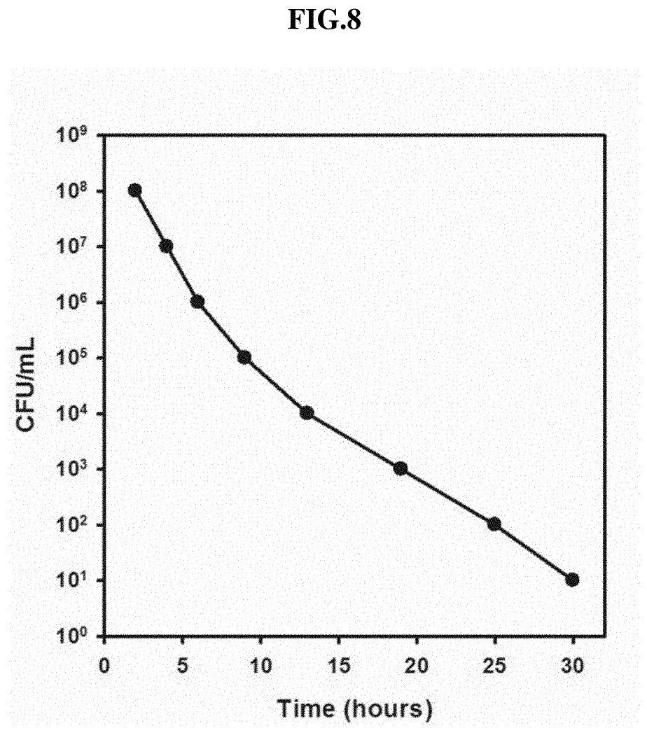

FIG. 8 is a graph illustrating the semi-quantitative results of the E. coli ampoule test. As can be seen in the graph, a negatively proportional correlation between the elapsed time (x-axis) of the appearance of the blue fluorescence and viable cell density (CFU/mL) of E. coli ATCC 25922 in the liquid sample (y-axis) is presented. The eight ampoules used in the semi-quantitative tests are prepared by filling mixed viable cell suspension of E. coli ATCC 25922 in the E. coli ampoule containing 1-20% of yeast extract, 10-40% of peptone, 10-40% of sodium chloride, 1-10% of lab-lemco powder, and 0.1-1% of 4-MUG. The above eight ampoules include different cell densities of mixed viable cell suspension of E. coli ATCC 25922 and are incubated 37° C. until respective eight ampoules show positivity of the inoculated bacteria (i.e. until blue fluorescence under the long-wave UV light is observed). After the measurement of the elapsed time, ampoules are removed from an incubator. Each point on the graph represents a test result of each of the eight ampoules, and a viable cell density (CFU/mL) of E. coli ATCC 25922 in the liquid sample (y-axis) is measured using plate count taking the liquid sample from the original bacterial culture. Several dilutions are performed to prepare different cell densities for respective eight ampoules. Then, the elapsed time for each eight ampoules are recorded which starts from the time of inoculating bacteria into respective ampoules to the time of the first appearance of positivity (i.e. blue fluorescence) and plotted in x-axis of the graph in FIG. 8. The elapsed time of the tests ranges from 2 hours to 30 hours. Biological triplicates are prepared for each condition. Data variance is +/−1 hour.

The semi-quantitative results of the E. coli ampoule test in FIG. 8 are used to estimate cell density of the E. coli in the test samples by measuring the elapsed time of the samples and using the graph in FIG. 8. This estimation method is capable of indicating a viable cell density of the bacteria in CFU/mL (colony formation unit per milliliter) ranging from <10 to 10⁸ CFU/mL. For example, a method for the detection of E. coli bacteria in liquid sample begins by preparing the liquid test sample in the E. coli ampoule, incubating the E. coli ampoule and checking the fluorescence detection periodically (e.g. every hour) until blue fluorescence under long wave UV for respective samples is shown. Then, the time of the blue fluorescence appearance for the samples are recorded and the bacteria density (i.e. viable cell density (CFU/mL)) in the tested liquid samples are estimated using the negatively proportional correlation between the elapsed time (x-axis) and a viable cell density (CFU/mL) of E. coli ATCC 25922 in the liquid samples (y-axis) in FIG. 8. By finding a particular Y value from the graph in FIG. 8 corresponding to the recorded elapsed time for the self-filled vacuum ampoule, the viable cell density (CFU/mL) of the E. coli bacteria in the liquid sample is estimated. If a user only needs to know whether or not the sample contains E. coli, then, the user may perform the test and observe whether there is blue fluorescence under long wave UV once after 30 hours without checking the fluorescence detection every hour.

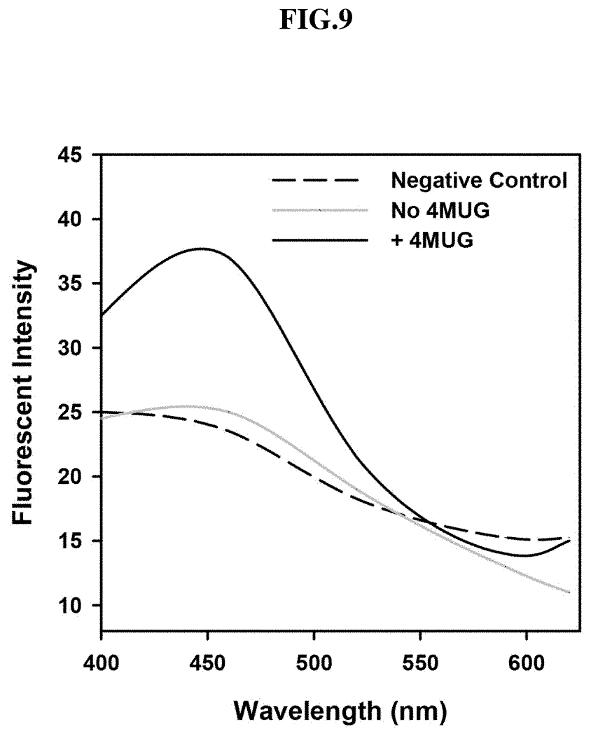

The formation of blue fluorescence in the E. coli ampoule test can also be captured and quantified using a fluorescent spectrometer. As shown in FIG. 9, the E. coli ampoule test is performed using 10² CFU/mL as the initial inoculation concentration with the presence of 4MUG. Three samples are prepared for the fluorescent spectrometer measurements. For the first sample, the E. coli ampoule test is performed using 10² CFU/mL as the initial inoculation concentration of E. coli ATCC 25922 with the presence of 4MUG. The ampoule for the first sample includes same nutrients for bacterial culturing used in the semi-quantitative tests of the E. coli ampoule in FIG. 8. For the second sample, in the negative control, no bacteria are inoculated. Other than the existence of bacteria inoculation, the nutrients for bacterial culturing and the detection reagents (4MUG) in the ampoule for the negative control are same with the fist sample. Instead of the bacteria inoculation, sterile milli-Q water with a neutral pH (7.0) is filled to the negative control. For the third sample, in the “No 4MUG” group, same bacterial concentration (i.e. 10² CFU/mL as the initial inoculation concentration) is applied without adding a detection reagent, 4MUG. After incubation of the three samples at 37° C. for 24 hours, the ampoule test mixtures are transferred to 96-well plate for the fluorescence measurement. The emission spectrums of E. coli ampoule tests with and without 4MUG are captured using the 355 nm excitation. Biological triplicates are used for each three samples. Fluorescence is measured by, for example, Molecule Device FlexStation 3 using the 355 nm excitation. Emission spectrum is scanned from 400 to 800 nm. Other means of fluorescence spectrometer may be employed by those having ordinary skill in the art.

In FIG. 9, an emission peak at 460 nm is observed only with the presence of 4MUG (“+4MUG”) but is not observed in “No 4MUG” and not in “Negative Control” groups. The formation of this emission peak at 460 nm is due to a reaction between E. coli and 4MUG which forms a fluorescent product. These results indicate that the fluorescent spectrometer can be used to quantitatively measure the positivity of the E. coli bacteria test result. The fluorescent spectrometer can detect the appearance of blue fluorescence of E. coli bacteria test result quicker than the visual fluorescence detection using a long-wave UV light.

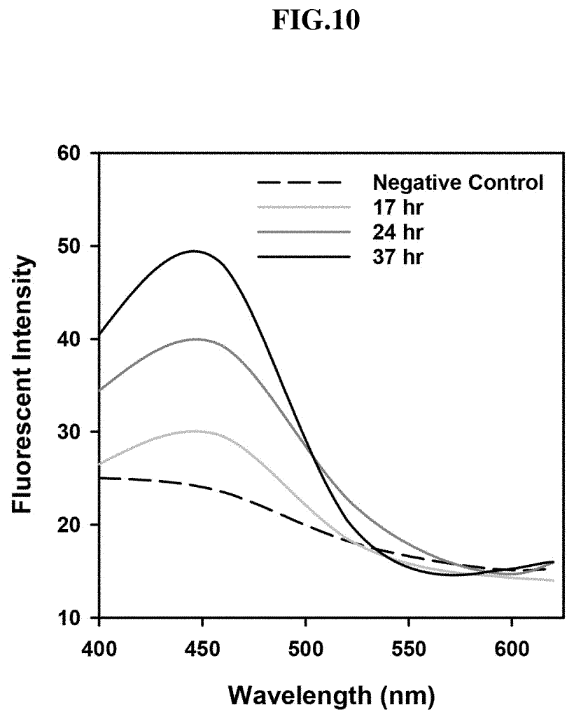

The appearance of the blue fluorescence is a result of an accumulative effect, i.e. as the reaction between bacteria and 4MUG (detection reagent) continues, the fluorescence will become more intense. This is confirmed by measurements of the fluorescent spectrum over time as shown in FIG. 10. Two samples (i.e. “+4MUG” group, and “Negative Control” group) used in the above fluorescent spectrum in FIG. 9 are prepared. Then, the fluorescent spectrum measurement for the “+4MUG” group is performed with different incubation time (e.g. 17 hours, 24 hours, 37 hours). After 17, 24 and 37 hours, the ampoule test mixture is transferred to the 96-well plate for the fluorescence measurement. The condition for the fluorescent spectrum measurement is same with the fluorescent spectrum measurement in FIG. 9.

As shown in FIG. 10, with same initial bacterial inoculation (i.e., 10² CFU/mL as the initial inoculation concentration of E. coli ATCC 25922 with the presence of 4MUG), the emission peak at 460 nm grows as the incubation time increases. On the other hand, the fluorescent spectrum measurement is performed for the “Negative Control” group but the emission peak at 460 nm is not observed. These results indicate that the fluorescence signal can be detected at an earlier time using fluorescence spectrometer. For example, with 10² CFU/ml initial bacteria density, about 25 hours incubation is needed to be able to observe the blue fluorescence using long-wave UV light, as compared with about 17 hours needed to be able to observe the blue fluorescence using fluorescence spectrometer.

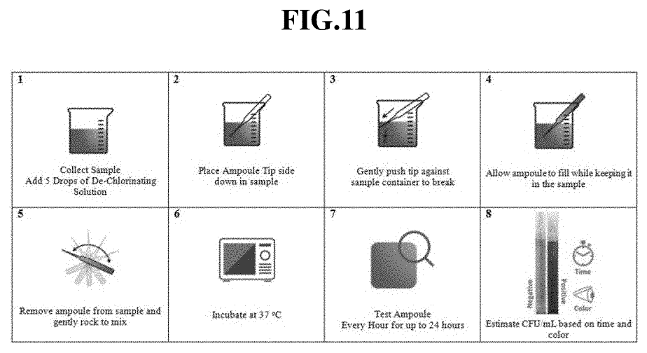

FIG. 11 shows a processing flow for conducting a bacteria test using the self-filling vacuum ampoule detection system in accordance with an embodiment of the present disclosure. A method to determine the specific bacteria type in liquid sample and estimate cell density of viable bacteria in liquid sample according to the embodiment illustrated in FIG. 11 includes processes using the results of a total viable bacteria ampoule test or an E. coli ampoule test according to the embodiments illustrated in FIGS. 1, 3, 4, and 8.

As shown in FIG. 11, in step 1 of the processes, a liquid sample to be tested is collected in a container. If chlorine is present in the collected liquid sample, de-chlorination liquid is added. In step 2, the ampoule tip, for example, the ampoule tip 3 of the vacuum ampoule total bacteria detection system in FIG. 1 to detect total viable bacteria or a tip of E. coli ampoule to detect E. coli bacteria is placed down in the collected liquid sample. The vacuum ampoule total bacteria detection system comprises a glass self-filling ampoule and an enclosed supporting medium and chemical indicator to detect total viable bacteria. The vacuum ampoule E. coli bacteria detection system comprises a glass self-filling ampoule and an enclosed supporting medium and chemical indicator to detect E. coli bacteria. In step 3, the ampoule tip is gently pushed against the container to break. In step 4, the vacuum ampoule is self-filled while keeping the ampoule in the collected liquid sample. In step 5, the self-filled vacuum ampoule is removed from the container and gently rocked and mixed.

In step 6, the self-filled vacuum ampoule is incubated at about 37° C. The incubation is performed until the first appearance of positivity of the viable bacteria is detected or up to about 30 hours. If the first appearance of positivity of the viable bacteria is not detected up to 30 hours, it indicates that the bacterial concentration in the collected liquid sample is below about 1 CFU/ml. In step 7, during the incubation, the self-filled vacuum ampoule is tested periodically (e.g. hourly) for up to 30 hours. That is, color change and color intensity are assessed every hour to determine the presence or amount of for example, total viable bacteria, or E. coli, and an elapsed time for the self-filled vacuum ampoule is recorded which starts from the beginning of incubation to the time of the first appearance of color change (e.g. pink-red color or blue fluorescent under long wave UV). The color change (e.g. pink-red color) is visually assessed to detect or quantify total viable bacteria or fluorescent blue color is assessed under long wave UV to detect or quantify E. coli.

In step 8, a viable cell density (CFU/mL) of the bacteria in the liquid sample is estimated based on the elapsed time and the color intensity. For example, the total viable bacteria density (i.e. viable cell density (CFU/mL)) in the tested liquid sample are estimated using the negatively proportional correlation between the elapsed time (x-axis) of the appearance of the pink-red color and a viable cell density (CFU/mL) of E. coli ATCC 25922 and S. aureus ATCC 25923 (y-axis) in FIG. 3. By finding a particular Y value from the graph in FIG. 3 corresponding to the recorded elapsed time for the self-filled vacuum ampoule total bacteria detection system, the viable cell density (CFU/mL) of the bacteria in the liquid sample is estimated. For another example, a color intensity of the pink-red color of the self-filled vacuum ampoule total bacteria detection system is observed and the observed pink-red color is compared to the color gradient references in FIG. 4 to estimate what is the total viable bacteria density (i.e. viable cell density (CFU/mL)) in the tested liquid sample. For another example, the color change can be detected using the fluorescence detection under the long-wave UV light periodically (e.g. every hour) until blue fluorescence for the self-filled vacuum ampoule is shown. Then, the bacteria density of E. coli (i.e. viable cell density (CFU/mL)) in the tested liquid sample are estimated using the negatively proportional correlation between the elapsed time (x-axis) of the appearance of the blue fluorescence and a viable cell density (CFU/mL) of E. coli ATCC 25922 (y-axis) in FIG. 8. By finding a particular Y value from the graph in FIG. 8 corresponding to the recorded elapsed time for the self-filled vacuum ampoule, the viable cell density (CFU/mL) of the E. coli bacteria in the liquid sample is estimated.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. A self-filling vacuum ampoule detection system to quantify viable bacteria in a liquid sample, the self-filling vacuum ampoule detection system comprising:
a vacuum ampoule including:
a supporting medium, wherein the supporting medium comprises nutrients for culture bacterial species in the liquid sample;
at least one selective reagent to inhibit a growth of interference microbial species in the liquid sample; and
a detection reagent to quantify an amount of bacterial species in the liquid sample,
wherein when the vacuum ampoule is self-filled with the liquid sample, and the viable bacteria is present in the liquid sample and react with the detection reagent, the self-filled vacuum ampoule is configured to change color.

2. The self-filling vacuum ampoule detection system of claim 1,
wherein the supporting medium comprises yeast extract, peptone, sodium chloride, or lab-lemco powder, and
wherein the detection reagent comprises 2,3,5-triphenyltetrazolium chloride (TTC) to detect the viable total bacteria in the liquid sample.

3. The self-filling vacuum ampoule detection system of claim 1,
wherein the vacuum ampoule comprises about 1˜20% of yeast extract, about 10˜40% of peptone, about 10˜40% of sodium chloride, about 1˜10% of lab-lemco powder, and about 0.1˜1% of 2,3,5-triphenyltetrazolium chloride (TTC), and detects the viable total bacteria in the liquid sample.

4. The self-filling vacuum ampoule detection system of claim 1,
wherein the supporting medium comprises yeast extract, peptone, sodium chloride, or lab-lemco powder, and
wherein the detection reagent comprises 4-Methylumbelliferyl-β-D-glucuronide hydrate (4-MUG) to detect E. coli bacteria in the liquid sample.

5. The self-filling vacuum ampoule detection system of claim 1,
wherein the vacuum ampoule comprises about 1˜20% of yeast extract, about 10˜40% of peptone, about 10˜40% of sodium chloride, about 1˜10% of lab-lemco powder, and about 0.1˜1% of 4-Methylumbelliferyl-β-D-glucuronide hydrate (4-MUG), and detects E. coli bacteria in the liquid sample.

6. A method to quantify viable bacteria in a liquid sample, the method comprising:
self-filling the liquid sample into a vacuum ampoule containing a supporting medium, at least one selective reagent, and a detection reagent, wherein the detection reagent comprises 2,3,5-triphenyltetrazolium chloride (TTC), and detects viable total bacteria in the liquid sample;
mixing the liquid sample with the supporting medium, the at least one selective reagent, and the detection reagent in the self-filled vacuum ampoule;
incubating the self-filled vacuum ampoule at about 37° C.;
during the incubating, observing a change of color of the self-filled vacuum ampoule, wherein the self-filled vacuum ampoule is configured to change color when the viable bacteria are present and react with the detection reagent;
measuring an elapsed time from the beginning of incubation to time at the change of color of the self-filled vacuum ampoule;
observing the changed color of the self-filled vacuum ampoule;
comparing the changed color of the self-filled vacuum ampoule with color gradient references; and
determining a viable cell density of the bacteria in the liquid sample based on a predetermined negatively proportional correlation between the elapsed time and the viable cell density of the bacteria, and further based on the comparison of the changed color with the color gradient references.

7. The method of claim 6, wherein the observing of the change of color of the self-filled vacuum ampoule is performed at predetermined intervals.

8. The method of claim 6, wherein the observing of the change of color of the self-filled vacuum ampoule is performed by observing appearance of pink-red color to detect the total viable bacteria.

9. The method of claim 6, wherein the observing of the change of color of the self-filled vacuum ampoule is performed by observing appearance of blue fluorescence under the long-wave UV light to detect E. coli bacteria.

10. (canceled)

11. (canceled)

12. (canceled)

13. The method of claim 6,
wherein the supporting medium comprises yeast extract, peptone, sodium chloride, or lab-lemco powder.

14. The method of claim 6, wherein the vacuum ampoule contains about 1˜20% of yeast extract, about 10˜40% of peptone, about 10˜40% of sodium chloride, about 1˜10% of lab-lemco powder, and about 0.1˜1% of 2,3,5-triphenyltetrazolium chloride (TTC), and detects the viable total bacteria in the liquid sample.

15. The method of claim 6,
wherein the supporting medium comprises yeast extract, peptone, sodium chloride, or lab-lemco powder, and
wherein the detection reagent comprises 4-Methylumbelliferyl-β-D-glucuronide hydrate (4-MUG), and detects E. coli bacteria in the liquid sample.

16. The method of claim 6,
wherein the vacuum ampoule contains about 1˜20% of yeast extract, about 10˜40% of peptone, about 10˜40% of sodium chloride, about 1˜10% of lab-lemco powder, and about 0.1˜1% of 4-Methylumbelliferyl-β-D-glucuronide hydrate (4-MUG), and detects E. coli bacteria in the liquid sample.

17. The method of claim 6, wherein the color gradient references comprise a plurality color image blocks which represent respective colors of sample vacuum ampoules with different viable cell densities of the bacteria.
